(19) United States
(12) Patent Application Publication
(10) Pub. No.: US 2006/0217695 A1
(43) Pub. Date: Sep. 28, 2006

(54) Optically-induced treatment of internal tissue

(76) Inventors: Leonard C. DeBenedictis, Palo Alto, CA (US); Barry G. Broome, Carlsbad, CA (US); George Frangineas, Fremont, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 11/354,217

(22) Filed: Feb. 13, 2006

Related U.S. Application Data
(63) Continuation-in-part of application No. 11/158,907, filed on Jun. 20, 2005.
Application No. 11/158,907 is a continuation-in-part of application No. 10/750,790, filed on Dec. 31, 2003.
(60) Provisional application No. 60/677,682, filed on May 3, 2005.
(60) Provisional application No. 60/652,891, filed on Feb. 14, 2005.
(60) Provisional application No. 60/652,891, filed on Feb. 14, 2005.

(51) Int. Cl. A61B 18/18

(52) U.S. Cl. 606/18; 606/13; 606/14; 606/17; 606/2

(57) ABSTRACT

An optical beam is delivered to internal target tissue, for example via two counter-rotating disks or via a single rotatable component. In one approach, two counter-rotating disks deflect an incident optical beam in a manner that generates an irradiation pattern at the target tissue. In another approach, a rotatable component includes a plurality of deflection sectors arranged around a rotation axis, and each sector deflects an incident optical beam as the sector rotates through the beam to generate an irradiation pattern at the target tissue. A probe maintains an optical channel within the human body so that the deflected optical beam can be delivered to the target tissue.

### CROSS-REFERENCE TO RELATED APPLICATION(S)

This application (a) claims priority under 35 U.S.C. § 119(e) to U.S. Provisional Patent Application Ser. No. 60/677,682, “Optically-Induced Treatment of Internal Tissue,” filed May 3, 2005; (b) claims priority to U.S. Provisional Patent Application Ser. No. 60/652,891, “High Efficiency, High Speed Optical Pattern Generator Using a Single Rotating Component,” filed Feb. 14, 2005; and (c) is a continuation-in-part of U.S. patent application Ser. No. 11/158,907, “Optical Pattern Generator Using a Single Rotating Component,” filed Jun. 20, 2005, which is a continuation-in-part of U.S. patent application Ser. No. 10/750,790, “High Speed, High Efficiency Optical Pattern Generator using Rotating Optical Elements,” filed Dec. 31, 2003, and which also claims priority to U.S. Provisional Patent Application Ser. No. 60/652,891, “High Efficiency, High Speed Optical Pattern Generator Using a Single Rotating Component,” filed Feb. 14, 2005. The subject matter of each of the foregoing is incorporated herein by reference in its entirety for all purposes.

### BACKGROUND OF THE INVENTION

1. Field of the Invention

This invention relates generally to the treatment and/or diagnostics of internal tissue (e.g., tissue located within the human body) by optical irradiation, for example via the delivery of laser irradiation to internal tissue.

2. Description of the Related Art

Lasers and other intense light sources can be used for various types of tissue treatment, including the treatment of internal tissues. Some examples include laser thoracic surgery, laser bowel surgery, laser brain surgery, and laser photo-coagulation for bowel, brain and ophthalmic (retinal) surgery. In many of these treatments, an optical beam irradiates the target tissue. If the underlying physical mechanism is based on absorption, then the optical beam typically operates at a wavelength that is absorbed by the target tissue. In the case where water is the primary absorbing material, cellular and interstitial water absorbs the optical energy and transforms it into thermal energy. The thermal energy increases the temperature of the tissue, causing the desired beneficial effect. For example, it may be desirable to heat the target tissue to the point of damage and then allow recovery by neighboring tissue. In most cases, it is desirable not to damage tissue underlying or surrounding the target tissue. Not only does this avoid unwanted damage, but it can also speed the recovery process. Denaturation of tissues containing collagen fibers can produce a beneficial effect of tissue shrinkage. In addition, tissues containing pools of blood such as hemorrhoidal tissue can be photo-coagulated and subsequently eliminated.

One difference between the treatment of internal tissue and the treatment of external tissue (e.g., skin) is that internal tissue is more difficult to access. The optical beam must be transported inside the body to the target tissue. As a result, the optical channel to the target tissue typically is much more confined since it is limited by the anatomy of the body. The optical channel is often narrow and long in order to reach the target tissue location within the body. In addition, the channel may also have thermal or other restrictions that limit the amount of waste heat that can be dissipated or the amount of physical debris that can be removed, for example. It may also be difficult to control the position or movement of the treatment device, to determine the position of the device relative to the target tissue or to obtain feedback about the efficacy of the treatment.

Currently, many laser technologies for tissue treatment use a single high power laser beam that is manually moved from one location to another at the target tissue, or that sweeps out a treatment path as the laser beam is scanned across the target tissue. However, this approach has many drawbacks. For example, high power laser beams are inherently more dangerous and can cause more damage if delivered to the wrong location. Traditional systems also are typically based on more expensive types of lasers and do not take advantage of lower power, cheaper semiconductor lasers. The delivery optics must also be designed to handle higher powers, further increasing the cost and complexity of the system. In addition, if the single laser beam must be manually moved to different locations on the target tissue, this can result in long treatment times.

Thus, there is a need for devices and methods that can direct an optical beam to generate an irradiation pattern at an internal target tissue, preferably overcoming some or all of the drawbacks discussed above.

### SUMMARY OF THE INVENTION

The present invention overcomes limitations of the prior art by delivering an optical beam to internal target tissue. In one approach, an apparatus for delivering an optical beam to target tissue within a human body includes two counter-rotating disks and a probe. The counter-rotating disks deflect an incident optical beam in a manner that generates an irradiation pattern at the target tissue. The irradiation pattern can be used for different purposes. For example, in some applications, it may be absorbed by the tissue, resulting in beneficial effects. In other applications, it may be used to irradiate the tissue for diagnostic purposes. The probe maintains an optical channel within the human body so that the deflected optical beam can be delivered to the target tissue.

Examples of probes include endoscopic, arthroscopic and catheter-based probes. The probe can enter the human body through a natural orifice or through an artificial orifice, such as an incision. In one approach, the probe includes an optical window that is in direct contact with the target tissue and the optical beam passes through the optical window to treat the target tissue.

In one embodiment, the apparatus generates an annular pattern of discrete spots. The counter-rotating disks contain pairs of corresponding facets. As the facets rotate through the incident optical beam, each pair of facets deflects the optical beam to one of the spots in the annular pattern. In a specific design, the apparatus includes a pyramidal polygon having N facets, where N is the number of pairs of facets on the disks. Each pair of facets on the disks deflects the incident optical beam to a corresponding facet on the pyramidal polygon, which in turn deflects the beam to one of the spots in the annular pattern.

One advantage of using counter-rotating disks is that the facets can be individually designed. Hence, irregular and non-planar irradiation patterns can be implemented. Another advantage is that the disks can be rotated at high speeds, resulting in fast treatment times.

In another aspect of the invention, an optical pattern generator (including approaches other than counter-rotating disks) directs an optical beam to an irradiation pattern that creates a plurality of microscopic treatment zones at the internal target tissue separated by untreated target tissue. A probe maintains an optical channel within the human body for delivery of the optical beam to the target tissue. The optical pattern generator may be located either internal or external to the human body.

In one variation, the probe includes an optical window that is in direct contact with the target tissue and the optical window is thermally conductive to facilitate heating or cooling of the target tissue. In other aspects of the invention, control logic can adjust various operational parameters in response to feedback from sensors. Examples include controlling the optical beam and/or the optical pattern generator based on the motion of the probe; controlling optical beam parameters such as wavelength, power, pulse duration, pulse energy, pulse shape, beam profile, duty cycle and pulse repetition rate; and controlling focus parameters such as numerical aperture, focal length and location of focus of the optical beam. The parameters can be adjusted adaptively during the course of treatment, or adjusted before treatment or before a series of treatments but held constant during treatment.

In still another aspect of the invention, the apparatus for delivering an optical beam to target tissue within the human body includes a rotatable component having a plane of rotation and a rotation axis. The rotatable component includes a plurality of deflection sectors arranged in a pattern around the rotation axis. Each sector deflects an incident optical beam as the sector rotates through the beam to generate a predetermined irradiation pattern at the target tissue. The apparatus also includes a probe for maintaining an optical channel within the human body for delivering the deflected optical beam to the target tissue.

In some embodiments, the rotatable component deflects the incident optical beam by a substantially constant angular deflection that is primarily in the plane of rotation of the rotatable component. In some variations, the rotatable component further includes a plurality of discrete structures arranged approximately around the rotation axis. In these embodiments, each discrete structure has at least two reflective faces, and reflective faces from adjacent structures form opposing reflective surfaces for the deflection sectors.

Other aspects of the invention include methods and systems corresponding to the devices and apparatus described above.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a perspective view of a device according to the present invention.

FIGS. 2A-2C are a perspective view, top view and head-on view of the optical train of the device in FIG. 1.

FIG. 3A is a head-on view and top view illustrating the 9:00 facets on the counter-rotating disks.

FIG. 3B is a head-on view and side view illustrating the 6:00 facets on the counter-rotating disks.

FIG. 4 is a block diagram showing a control system for a device according to the invention.

FIG. 5 is a perspective view of a galvanometer-based device according to the present invention.

FIG. 6 is a perspective view of a spatially multiplexed holographic-based device according to the present invention.

FIG. 7 is a perspective view of a device with a single rotating component according to the present invention.

FIG. 8 is a side view of the rotating component of FIG. 7 according to the invention, where the incident optical beam lies substantially in the plane of rotation.

FIG. 9 is a close-up view showing tilting of the prisms of FIG. 8.

FIG. 10 is a side view illustrating the principle of operation of another optical pattern generator according to the invention, where the incident optical beam has a substantial component in a direction normal to the plane of rotation and the full parent surfaces of the reflective segments are shown.

### DETAILED DESCRIPTION OF THE PREFERRED EMBODIMENTS

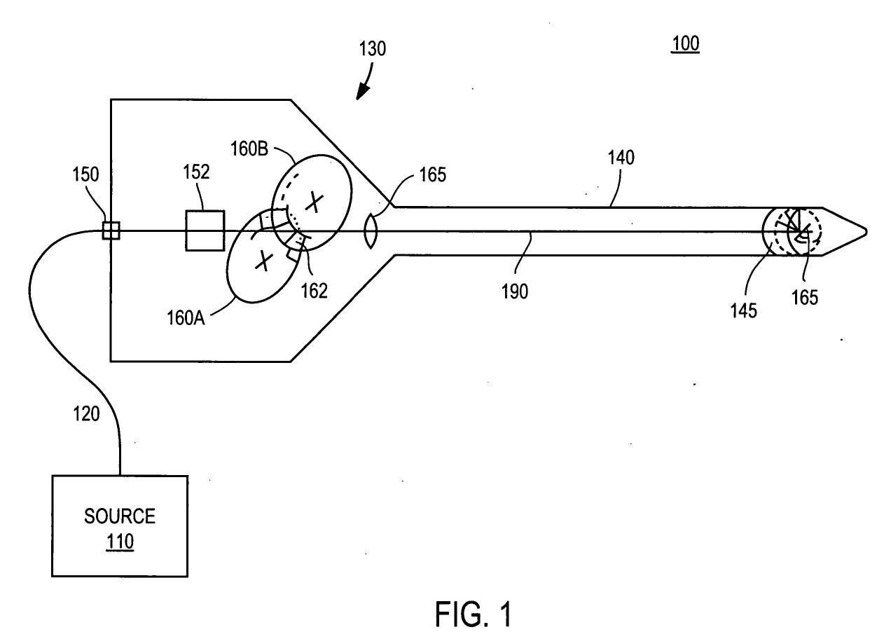

FIG. 1 is a perspective view of a system 100 according to the present invention. The system 100 includes an optical source 110, an optical fiber 120 and a handpiece 130. The handpiece 130 includes a probe portion 140 used for insertion into the human body. The optical train within the handpiece 130 includes an input port 150 for the fiber, collimating optics 152, two counter-rotating disks 160A-B, and additional delivery optics 165. The probe 140 includes a transparent window 145 located in the vicinity of the probe's tip. In this example, the window 145 is located on the cylindrical side of the probe 140.

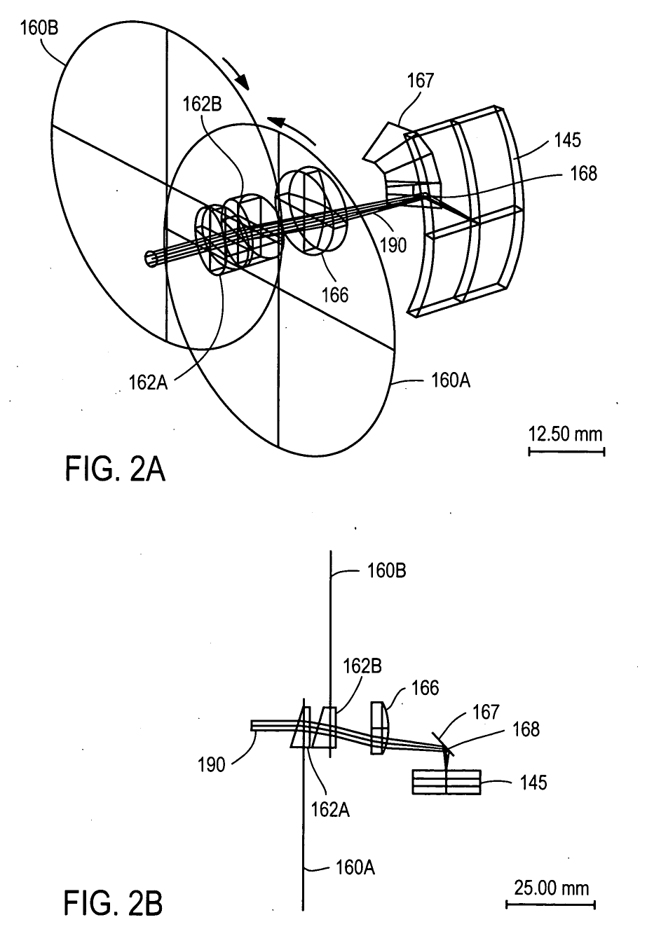

The system 100 operates as follows. The optical source 110, for example a pulsed laser, generates an optical beam that is delivered to the handpiece 130 via the fiber 120. The optical beam enters the handpiece at input port 150 and is collimated by optics 152 (e.g., a collimating lens). The counter-rotating disks 160 shown in FIGS. 2A-2C each include many facets 162 arranged around their periphery. As the disks 160 rotate, different facets 162 travel through the optical beam 190, causing deflections to different directions. The deflected beam 190 is guided by the delivery optics 165 to the exterior of the transparent window 145. Rotation of the disks 160 generates an irradiation pattern at the transparent window 145. In this particular example, the pattern is an annular pattern. In other words, rotation of the disks 160 produces a series of spots arranged in a circle centered on the optical axis.

When the probe 140 is inserted into the human body, the window 145 is positioned to contact the target tissue. The rotating disks 160 then generate an annular irradiation pattern at the target tissue. The probe can also provide mechanical integrity and can thus maintain an optical channel within the body for delivery of the optical beam 190 to the target tissue.

Different irradiation patterns can be used, depending on the application and anatomy. For example, a continuous laser beam can be scanned across the target tissue. Alternately, the optical beam might be pulsed. The probe 140 can be positioned and then the laser pulsed to irradiate one particular location, and the probe 140 repositioned and the laser pulsed again, etc.

In the example of FIG. 1, the irradiation pattern is used for treatment of the target tissue and creates a large number of microscopic treatment zones separated by untreated target tissue. The optical beam is directed to each of the treatment locations but is held fairly stationary at that location until it “jumps” to the next location. The treatment zones are microscopic in the sense that they are not macroscopic in size. For example, see U.S. patent application Ser. No. 10/888,356, “Method and Apparatus for Fractional Photo Therapy of Skin,” by DeBenedictis et al., which is incorporated by reference herein. A large number of relatively small “microscopic” treatment zones separated by untreated areas can be used to effect treatment of the target tissue, as opposed to exposing the entire target tissue to a single high power optical beam. The term microscopic treatment zone is not meant to imply that the treatment zones must be so small that they are only visible under a microscope.

The specific design of the device 100 depends on the application and anatomy. For example, the source 110 typically is a laser source, although non-laser sources can also be used. Flash lamps and light-emitting diodes are examples of non-laser sources. Examples of laser sources include lasers, diode-pumped solid state lasers, Er:YAG lasers, Nd:YAG lasers, Er:glass lasers, argon-ion lasers, He—Ne lasers, carbon dioxide lasers, excimer lasers, fiber lasers such as erbium fiber lasers, ruby lasers, frequency multiplied lasers, Raman-shifted lasers, optically-pumped semiconductor lasers, pulsed dye lasers, and so forth. Both continuous and pulsed sources may be used, depending on the application. The optical source 110 could include one particular type of laser capable of providing one wavelength (or wavelength range) or tunable to different wavelengths. Alternatively, the optical source 110 could include two or more different types of lasers to provide a variety of different wavelengths or wavelength ranges. Optical beams from different light sources can be directed to the target tissue on a one-by-one basis or simultaneously.

The wavelength of the optical beam also depends on the application. The optical beam preferably has a wavelength between approximately 180 nm and 12,000 nm, such as between approximately 500 nm and 3,000 nm, or preferably between about 1000 nm and about 2000 nm, or more preferably between about 1400 nm and about 1600 nm. Terms such as “optical” and “light” are meant to include all of these wavelengths and are not meant to be limited to the visible.

In certain applications, the optical energy is intended to be absorbed by the human tissue. The absorption in human tissue in the visible light range is mostly due to specific chromophores (such as hemoglobin or melanin). In the near-infrared radiation range, water is typically a significant chromophore. The absorption coefficient for water in the near infrared range has peaks near 1450 nm (i.e. absorption coefficient of about 30/cm) and 1930 nm (i.e. absorption coefficient of about 130/cm) and between these peaks it does not drop below approximately 5/cm. Above the peak at 1930 nm, the absorption does not drop to small values but increases to extremely high values comparable to the absorption of Er:YAG laser light and/or CO₂ laser light. Between 1000 nm and 1450 nm the absorption coefficient decreases steadily and can be as low as 0.2/cm or less. Below about 1000 nm, chromophores such as hemoglobin and melanin (if present) become more prevalent and water absorption recedes.

Referring now to the handpiece 130, the shape and construction of the probe portion also depends on the application. Candidate applications include, for example, the treatment of hemangiomas; laryngeal or intra-anal warts; cervical, colon and other space occupying cancers; nasal polyps; lichen sclerosus of the vagina; hemorrhoids; and obstructive sleep apnea (palatal insufficiency). Endoscopes, arthroscopes and catheters are common probe types that may be suitable depending on the application. The probe 140 can enter the body through a natural orifice (anus, mouth, etc.) or through an “artificial” orifice (e.g., a surgical incision).

The window 145 in FIG. 1 is located on the cylindrical side of the probe 140 (as opposed to on the end of the probe). In alternate embodiments, it can be located at other positions, including at the end of the probe. It can also be differently shaped and more than one window can be used. If temperature control is desirable, sapphire or diamond windows may be used for their high thermal conductivity and transparency to pertinent wavelengths. Active heating or cooling can be implemented through the windows 145 or through other sections of the probe.

In the example of FIG. 1, the rotating disks 160 are designed to remain outside the human body when the probe 140 is inserted. However, in other implementations, the optics that generates the irradiation pattern may be located within the probe if they are small enough (or if the probe is large enough). One advantage to locating the optical pattern generator within the probe (e.g., at the probe tip) is that the remainder of the probe can be made more flexible. For example, the probe may contain a fiber that delivers the optical beam to the tip, where the irradiation pattern is then generated. In contrast, the probe 140 in FIG. 1 is designed to maintain a fixed free space optical path from the rotating disks 160 to the exit window 145, possibly limiting the flexibility of the probe 140.

Different irradiation patterns can also be implemented. Device 100 generates an irradiation pattern of equally spaced spots arranged in an annulus around the probe 140. Multiple annuli can also be generated (e.g., by using multiple sources, each source generating one of the annuli or by using a beamsplitter to generate multiple source beams). If the annuli all have the same diameter, then the irradiation pattern will be cylindrical. If the diameters are increasing or decreasing, then the irradiation pattern will be conical. Other non-planar patterns can also be generated. One advantage of the rotating disk approach is that irregular patterns may be supported. Each pair of corresponding facets 162 may be independently designed to produce a different spot within the irradiation pattern.

In one approach, the probe portion 140 is detachable from the rotating disk 160 portion. Different probes may be used for different applications and/or simply to accommodate different size anatomies.

FIGS. 2A-2C show different views of the optical train of device 100. These figures show the two counter-rotating disks 160 and the delivery optics, which includes a focusing lens 166 and a multi-faceted pyramidal polygon 167 (only a portion of which is shown). The cylindrical section 145 is the window. Corresponding facets 162 on the disks 160 are also shown. FIG. 2 shows one facet 162A on disk 160A and the corresponding facet 162B on disk 160B. In this example design, there are a total of twelve facets 162 on each disk 160, and there are also twelve facets 168 on the pyramidal polygon 167. Each pair of corresponding facets 162 on the disks also has a corresponding facet 168 on the pyramidal polygon 167. For convenience, these facets will be referred to as the 1:00, 2:00, etc. facets, referring to the relative clock location of the final spot on the window 145, from the frame of reference of a viewer located at the source (i.e., to the left of FIGS. 2A and 2B).

In FIG. 2, the 3:00 facets are active. The optical beam 190 travels through the pair of 3:00 facets 162A-162B. Note that the 3:00 facets need not be located at the 3:00 position on their respective disks; the 3:00 label refers to the final spot on the window 145. These facets 162 deflect the optical beam 190 to the 3:00 facet 168 on the pyramidal polygon 167, which reflects the optical beam to the 3:00 position at window 145. The focusing lens 166 brings the optical beam 190 to a focus around the exterior side of window 145 (e.g., at or slightly below the surface of the target tissue).

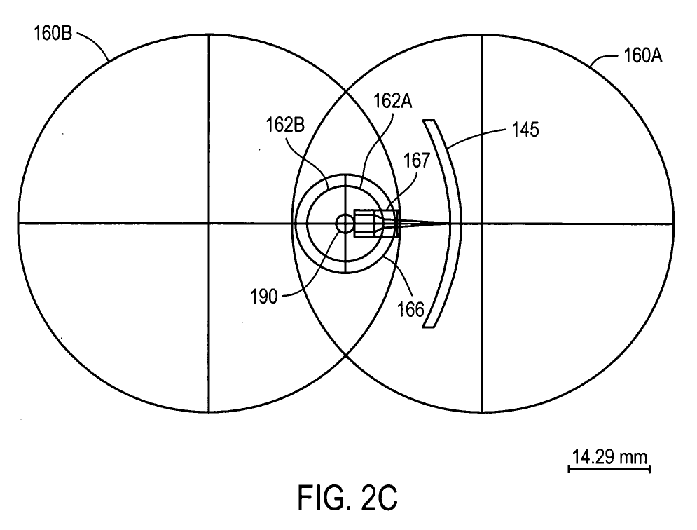

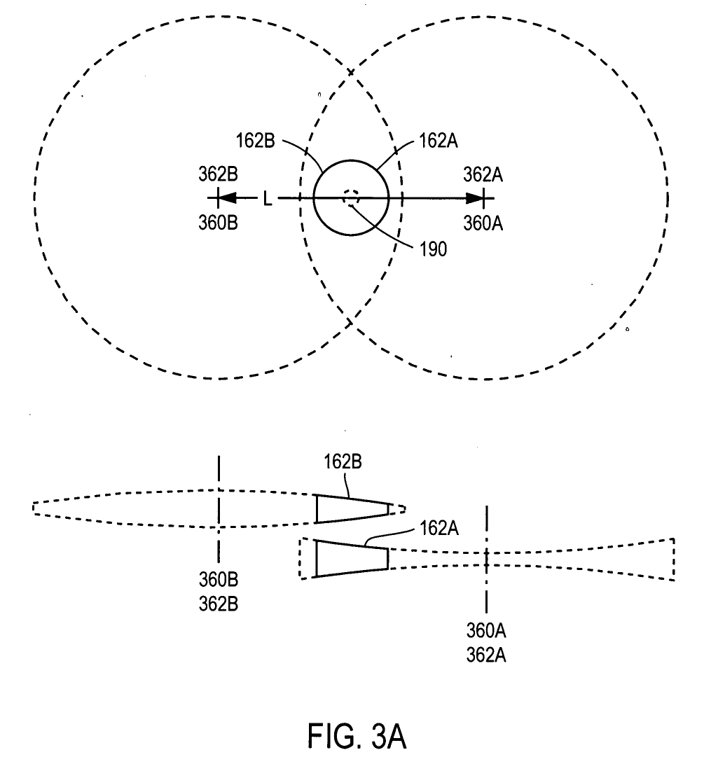

FIGS. 3A-3B further describe the facets 162 for the 9:00 position. In these figures, the marks 360A-360B are the centers of rotation of the two disks 160A-160B. The distance between these two centers is denoted by L. The disks themselves are not shown. To first order, the facets 162A-162B are lenses whose optical centers are located at 362A-362B, respectively, as represented by the marks 362A-362B and the dashed circles centered on these marks. Facet 162A is a negatively powered lens and facet 162B is a positively powered lens. The separation between optical centers 362A-362B is also L, but the line connecting the optical centers 362A-362B may be at an angle relative to the line connecting rotational centers 360A-360B. In these figures, the facets are shown in their “neutral” position, which is the midpoint of the rotation of the facet through the optical beam 190. Note that the large dashed circles describe the “parent” optical element for each facet but do not show the physical extent of each facet. The physical extent is shown by the smaller solid circles marked 162A-162B.

FIG. 3A shows a head-on view of the 9:00 facets 162 and also shows a top view of these facets. The dashed lens outline in the top view describes the parent optical element for each facet; the physical extent is marked by the solid outline. In FIG. 3A, the optical beam 190 is deflected towards 9:00 by the first facet 162A and further deflected in the same direction by the second facet 162B. Since each facet 162A-162B has an optical center 362A-362B that is coincident with the corresponding rotational center 360A-360B, the optical effect does not change as the facets 162 rotate through the optical beam 190. Therefore, as long as the optical beam 190 is incident on the 9:00 facets, it is deflected towards the 9:00 position. A similar analysis holds for the 3:00 position, and similarly for other positions.

FIG. 3B shows the situation for the 6:00 position. Optically, when in the neutral position, the 6:00 facet 162A is a negative lens with optical center 362A and the 6:00 facet 162B is a positive lens with optical center 362B. As shown in the side view, this results in a deflection of the optical beam 190 towards the 6:00 facet of the pyramidal polygon 167 and then the 6:00 position on the window 145. However, one difference compared to FIG. 3A is that the optical centers 362A-362B are not coincident with the corresponding rotational centers 360A-360B. As a result, the optical centers 362A-362B will shift as the facets 162A-162B for succeeding image positions rotate through the optical beam.

If uncorrected, this shift typically will cause a slight orthogonal deviation in the deflection of the optical beam 190 (unlike the 3:00 and 9:00 cases). In some applications, the deviation may be small enough that no correction is required. In other cases, correction of this residual cross-scan angular displacement may be achieved by introducing some degrees of design freedom in the facets or the rest of the optics. For example, the optical centers 362A-362B of the facets may be decentered from their original positions or aspheric surfaces can be used on the facets, or different radii used on the pairs of facets, or other correction may be added to the facets themselves. Alternately, the focusing lens 166 and/or the 6:00 facet 168 on the pyramidal polygon 167 may be used for correction.

FIGS. 3A-3B illustrate the basic operation of the counter-rotating disks, but other variations will be apparent. For example, the number of facets can be changed to generate more or fewer spots. In addition, since the facets can be independently designed, many different irradiation patterns are possible. Rather than a regular spacing of spots that covers a full 360 degrees, the spots can be concentrated within one or more sectors. For example, the spots could be evenly spaced between the 10:00 to 2:00 positions. Alternately, half the spots could be located between 10:00 and 11:00 and the other half between 1:00 and 2:00. As another example, the spots need not be regularly spaced. They could span the 12:00 to 3:00 positions but with a denser concentration in the 12:00 to 1:00 region. Multiple facets could target the same spot, resulting in multiple irradiation of one treatment zone.

The spots can also be offset in the axial direction. The basic irradiation pattern can also be moved in the axial direction by translating the entire probe (either manually or automatically) or by moving certain optical elements within the probe. For example, the lens 166 plus pyramidal polygon 168 may be axially translated while the disks rotate, thus repeating the annular irradiation pattern at different axial locations. Other components can also be used to introduce additional scanning motions. For example, the pyramidal polygon may rotate or oscillate, or galvanometers may be used to introduce additional motion.

The facets and other devices in the optical train can also introduce effects other than pure optical power and scanning. Aspheric surfaces, choice of materials, and more complex optical designs (e.g., doublets, triplets, etc.) can be used to either correct or to intentionally introduce higher order wavefront deviations, thus providing greater control over the beam shape and direction at the treatment zone.

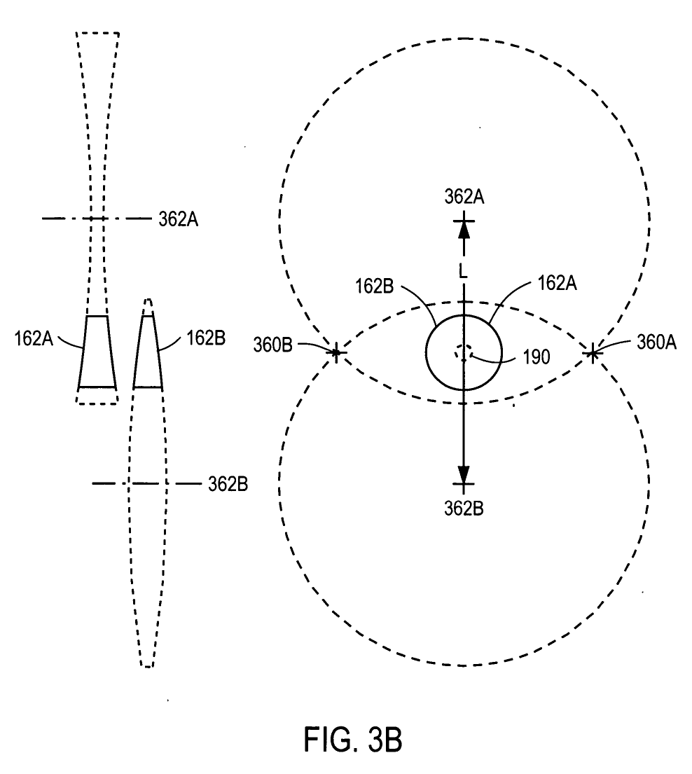

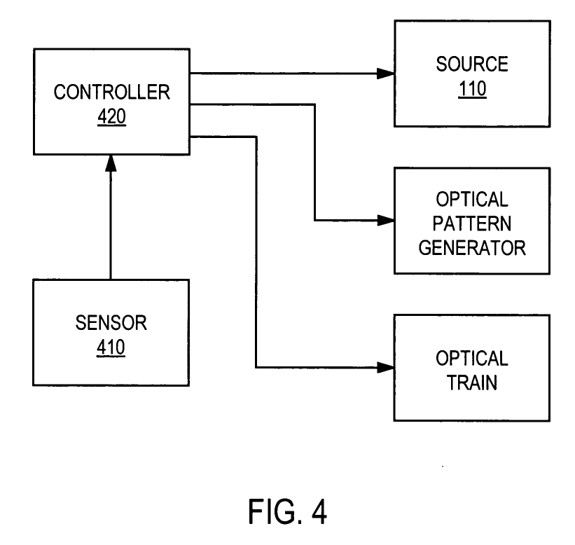

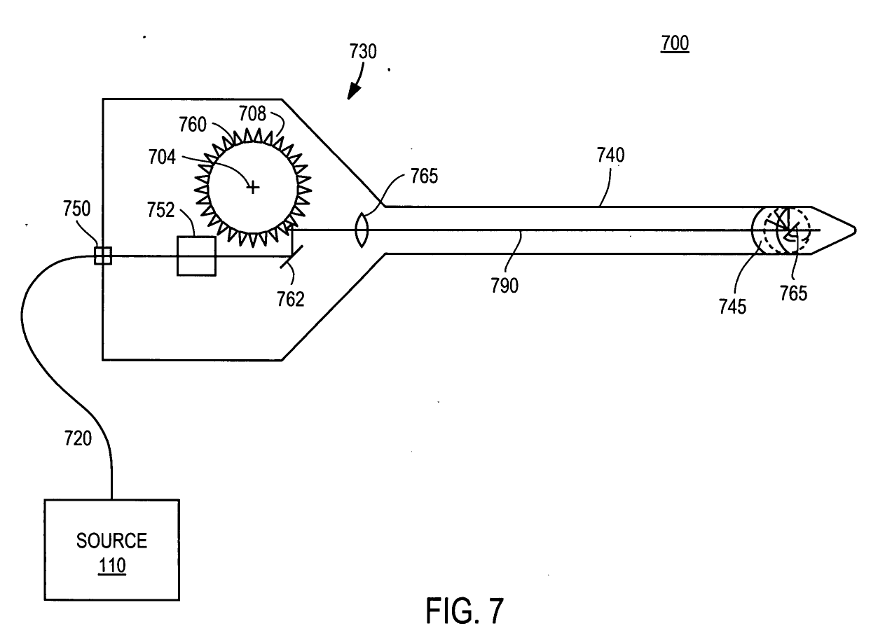

Additional examples concerning the design of counter-rotating disks are disclosed in U.S. patent application Ser. No. 10/750,790, “High speed, high efficiency optical pattern generator using rotating optical elements,” filed Dec. 31, 2003 by Len DeBenedictis et al., which is incorporated herein by reference. For example, FIG. 1B illustrates the use of multiple sources, FIG. 1C illustrates offset in one direction coupled with scanning in another direction, FIGS. 2-3 further illustrate offset along one direction, FIGS. 5A-5B illustrate a reflective design and FIGS. 7A-7C illustrate different types of spot patterns. Further examples are disclosed in U.S. patent application Ser. No. 10/914,860, “Two-dimensional optical scan system using a counter-rotating disk scanner,” filed Aug. 9, 2004 by Barry G. Broome et al., which is incorporated herein by reference. FIGS. 4-9 illustrate different designs that combine counter-rotating disks that produce offset in one direction with another device (e.g., galvanometer) that produces offset in the other direction.

As a result of this design freedom, many different types of irradiation pattern are possible. In a preferred embodiment, the irradiation pattern generates microscopic treatment zones separated by untreated target tissue. One advantage is that the neighboring untreated target tissue can speed recovery of the irradiated tissue, if so desired. In another application, rather than treating the target tissue, the irradiation pattern is used for diagnostic purposes.

In one approach, the optical beam is not continuous. Rather, it consists of separate optical pulses. The optical pulses can be generated by a pulsed laser. Alternately, a continuous optical beam can be converted into pulses by external components, for example by gating the output of a continuous wave laser or by inserting a chopper into the optical train at some point. Regardless of how they are produced, different optical pulses are then directed to different locations to create microscopic treatment zones. In one approach, a fixed number of fixed energy pulses (one or more) are delivered to each zone. In another design, the number of pulses and/or their energy can be adjusted, and then either held constant during the treatment or continuously adjusted during the treatment.

Other parameters can also be controlled. FIG. 4 is a block diagram of a control system for controlling various parameters. The control system includes sensor(s) 410 that are coupled to control logic 420. Control logic 420 can be used to control various parts of the overall system, including for example (in reference to FIG. 1) the optical source 110, the optical pattern generator 160 and/or other parts of the optical train 150, 152, 165. The control logic 420 adjusts the desired operational parameters based on feedback received from the sensor 410.

Examples of operational parameters include wavelength; energy, power, and energy and power density; pulse duration, pulse repetition rate, and temporal and spatial pulse shape; polarization; numerical aperture, depth of focus and location of focus; and angle of incidence on the target tissue. Aggregate operational parameters include number or density of optical pulses directed to each treatment zone; and the total energy deposited at each treatment zone. Parameters of the irradiation pattern may also be controlled, including for example separation between treatment zones, size of treatment zones and the spatial location of the treatment zones.

Examples of different types of sensors include optical coherence tomography, confocal microscopy, optical microscopy, optical fingerprinting and ultrasound. Tissue properties that may be measured include for example temperature, mechanical density, color, birefringence, opacity, absorption, extinction, scattering, albedo, polarizability, dielectric constant, capacitance, chemical balance, elastic properties, fractions of different materials (e.g., water, hemoglobin, oxyhemoglobin and foreign matter) and the properties of fluids introduced into the tissue. Probe position may also be used as feedback, including position, velocity and/or angular orientation.

Conventional control algorithms can be used to effect control based on feedback from the sensor. See also U.S. patent application Ser. No. 10/745,761, “Method and apparatus for monitoring and controlling laser-induced tissue treatment,” filed Dec. 23, 2003 by Leonard C. DeBenedictis and Thomas R. Myers, which is incorporated herein by reference and discloses especially control based on handpiece position. See also U.S. patent application Ser. No. 10/868,134, “Adaptive control of optical pulses for laser medicine,” filed Jun. 14, 2004 by David Eimerl and Leonard C. DeBenedictis, which is incorporated herein by reference and discloses especially control based on tissue properties.

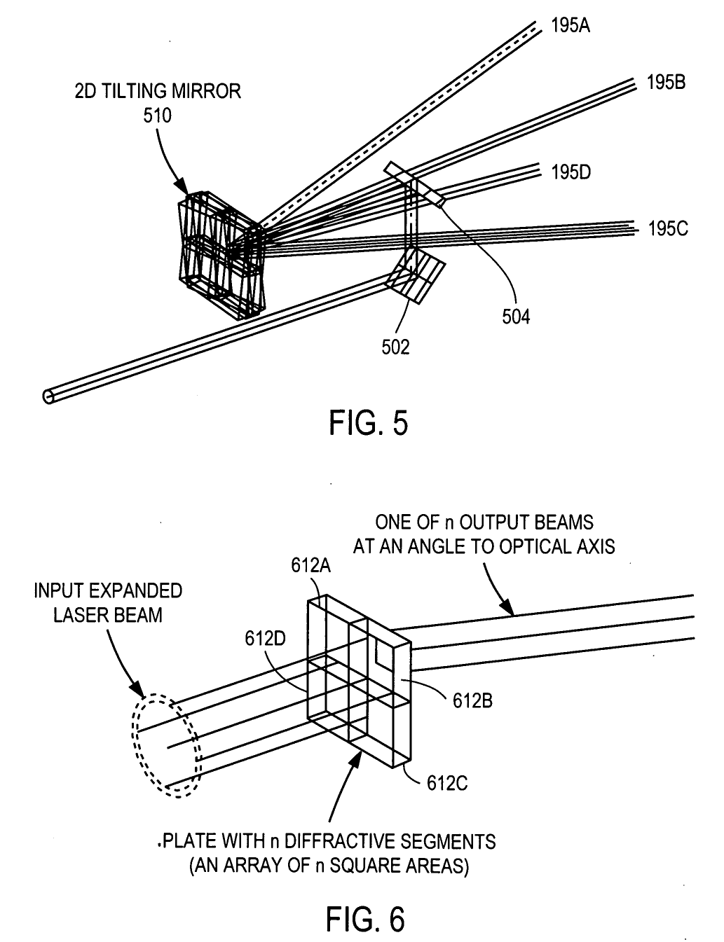

FIGS. 5-6 illustrate two example devices based on optical pattern generators other than counter-rotating disks. In FIG. 5, a two-dimensional tilting mirror 510 is used as the optical pattern generator. In this example, the incoming beam is directed to the tilting mirror 510 by turning mirrors 502 and 504. The motion of the tilting mirror 510 directs the optical beam to different locations in the irradiation pattern. FIG. 5 shows four different deflected beams 195A-195D. If the lens 166 plus pyramidal polygon 168 of FIG. 2 were used, the tilting mirror 510 could deflect the beam to each of the facets of the pyramidal polygon in sequence.

In FIG. 6, a spatially multiplexed holographic optical element or binary diffractive optical array element 610 is used as the optical pattern generator. The incoming optical beam is incident on the holographic optical element 610, which in FIG. 6 has four different spatial sections 612A-612D. Each section 612 deflects a portion of the optical beam to a different location (only one deflected beam is shown). In this example, there is no motion and all spots in the irradiation pattern are generated simultaneously. Referring to the pyramidal polygon of FIG. 2 again, the holographic optical element 610 could include twelve different sections, each of which directed a portion of the incoming optical beam to one of the twelve facets on the pyramidal polygon. Non-holographic spatially multiplexed devices can also be used as pattern generators (e.g., an array of lenses), as can non-spatially multiplexed holographic devices.

FIG. 7 is a perspective view of a system 700 with a single rotating component according to the present invention. The system 700 is designed similarly to the system 100 illustrated in FIG. 1, however the system 700 includes a single rotating component rather than the two counter-rotating disks 160A-B shown in FIG. 1. Similar to system 100, system 700 also includes an optical source 710, an optical fiber 720 and a handpiece 730. The handpiece 730 includes a probe portion 740 used for insertion into the human body. The optical train within the handpiece 730 includes an input port 750 for the fiber, collimating optics 752, a single rotating component 760, optics 762, and additional delivery optics 765. The probe 740 includes a transparent window 745 located in the vicinity of the probe's tip. In this example, the window 745 is located on the cylindrical side of the probe 740.

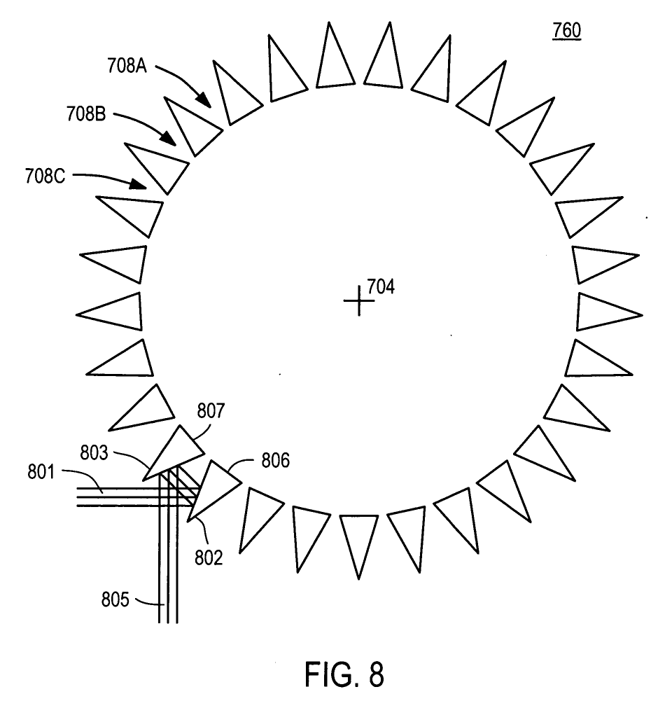

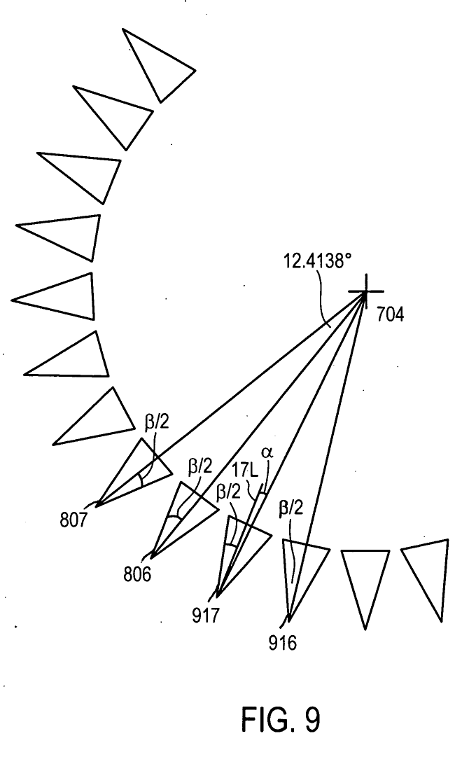

The system 700 operates as follows. The optical source 710, for example a pulsed laser, generates an optical beam that is delivered to the handpiece 730 via the fiber 720. The optical beam enters the handpiece at input port 750 and is collimated by optics 752 (e.g., a collimating lens). The rotating component 760 includes a number of sectors 708 arranged in a circle centered on a rotation axis 704 of the rotating component 760 (shown in more detail in FIGS. 8 and 9). The optical beam 790 propagates along a direction that lies in the plane of rotation. Each sector 708 includes a pair of reflective elements (e.g., reflective surfaces or a reflective coating). As the component 760 rotates, the sectors 708 rotate through the optical beam 790. Each sector 708 deflects the incoming optical beam 790 by some angular amount, as described in more detail below. The deflected beam 790 is guided by the delivery optics 765 to the exterior of the transparent window 745. Rotation of the component 760 generates an irradiation pattern at the transparent window 745.

Similar to system 100, when the probe 740 is inserted into the human body, the window 745 is positioned to contact the target tissue. The rotating component 760 then generates an irradiation pattern (e.g., an annular irradiation pattern) at the target tissue. The probe can also provide mechanical integrity and can thus maintain an optical channel within the body for delivery of the optical beam 790 to the target tissue.

As described above with regard to system 100, different irradiation patterns (e.g., annular patterns, irregular patterns, etc.) can be used, depending on the application and anatomy, and the optical beam can also be pulsed in some embodiments. In the example of FIG. 7, the irradiation pattern is used for treatment of the target tissue and creates a large number of microscopic treatment zones separated by untreated target tissue. The optical beam is directed to each of the treatment locations but is held fairly stationary at that location until it “jumps” to the next location. A large number of relatively small “microscopic” treatment zones separated by untreated areas can be used to effect treatment of the target tissue, as opposed to exposing the entire target tissue to a single high power optical beam.

The specific design of the device 700 depends on the application and anatomy, as described above with regard to system 100. For example, the source 710 can be a laser source, a non-laser source, a continuous or pulsed source, a laser capable of providing one wavelength or different wavelengths, two or more different types of lasers providing a variety of different wavelengths or wavelength ranges, and so forth. Additionally, the different applications, probe types (e.g., probe shapes and constructions), and probe entry methods described above regarding system 100 also apply to system 700. In one approach, the probe portion 140 is detachable from the rotating disk 160 portion. The rotating component 760 can be designed to remain outside the human body when the probe 740 is inserted, or the optics that generate the irradiation pattern can be located within the probe. With the optics inside the probe, the probe can be made more flexible, as described above. Furthermore, the window 745 in FIG. 7 can be located on the cylindrical side of the probe 740 or at other positions, it can be differently shaped, more than one window can be used, and so forth as described above.

The wavelength of the optical beam also depends on the application. The optical beam preferably has a wavelength between approximately 180 nm and 12,000 nm, such as between approximately 500 nm and 3,000 nm, or preferably between about 1000 nm and about 2000 nm, or more preferably between about 1400 nm and about 1600 nm. Similar to system 100, in certain applications, the optical energy for system 700 is intended to be absorbed by the human tissue. The absorption in human tissue in the visible light range is mostly due to specific chromophores (such as hemoglobin or melanin), and water is typically a significant chromophore in the near-infrared radiation range, as explained in more detail above

FIG. 8 is a side view of the rotating component, an optical pattern generator, according to the invention, where the incident optical beam 801 in FIG. 8 lies substantially in the plane of rotation of rotatable component 760. In this example, the rotating component 760 is divided into twenty nine sectors 708A, 708B, 708C, etc., which are arranged in a circle centered on the rotation axis 704 of the rotating component 760. The incident optical beam 801 propagates along a direction that lies in the plane of rotation. Each sector 708 includes a pair of reflective elements (e.g., reflective surfaces 802 and 803 for the sector that is currently active). The surface normals of the reflective surfaces have a substantial component in the plane of rotation. In this example, the rotating component 760 includes prisms 806, 807, etc. that are arranged in a circle. The faces of the prisms are reflectively coated and the reflectively coated surfaces from adjacent prisms (e.g., reflective surfaces 802 and 803 from prisms 806 and 807) form the opposing reflective surfaces for a sector. Discrete structures other than prisms can also be used and the reflective surfaces need not be planar. Small flat mirrors can be used in the place of the prism components.

As the component 760 rotates, the sectors 708 rotate through the incident optical beam 801. Each sector 708 deflects the incoming optical beam 801 by some angular amount. The sectors 708 are designed so that the angular deflection is approximately constant as each sector rotates through the incident optical beam 801, but the angular deflection may vary from sector to sector. In more detail, the incident optical beam 801 reflects from the first reflective surface 802 on prism 806, and subsequently reflects from reflective surface 803 on prism 807 before exiting as output optical beam 805.

The two reflective surfaces 802 and 803 form a Penta mirror geometry. An even number of reflective surfaces that rotate together in the plane of the folded optical path has the property that the angular deflection is invariant with the rotation angle of the reflective surfaces. In this case, there are two reflective surfaces 802, 803 and rotation of the disk 760 causes the prisms 806, 807 and their reflective surfaces 802, 803 to rotate together in the plane of the folded optical path. As a result, the output beam angle 805 does not change as the two reflective surfaces 802, 803 rotate through the incident optical beam 801. The reflective surfaces 802, 803 are self-compensating with respect to rotation of the disk 760. Furthermore, if the reflective surfaces 802, 803 are planar, they will also be substantially spatially invariant with respect to disk wobble.

As the disk 760 rotates clockwise to the next sector 708 and the next two reflective surfaces, the angular deflection can be changed by using a different included angle between the opposing reflective surfaces. For this configuration, the beam will be deflected by an angle that is twice that of the included angle. For example, if the included angle for sector 708A is 45 degrees, sector 708A will deflect the incident optical beam by 90 degrees. If the included angle for sector 708B is 44.5 degrees, then the incident optical beam will be deflected 89 degrees, and so on. In this example, different included angles are used for each of the sectors so that each sector will produce an output optical beam that is deflected by a different amount. However, the deflection angle will be substantially invariant within each sector due to the even number of reflective surfaces rotating together through the incident beam. For this example, the angular deflections have a nominal magnitude of 90 degrees and a variance of −15 to +15 degrees from the nominal magnitude.

In this example, the apex angle of each prism is 32.5862 degrees, calculated as follows. Each sector 708 subtends an equal angular amount. Since there are twenty nine sectors, each sector subtends 360/29=12.4138 degrees as shown in FIG. 9. The two prisms 806 and 807 have the same shape and, therefore, the same apex angle β. The component 760 is designed so that when the included angle is 45 degrees, the prisms 806 and 807 are positioned so that the line that bisects the apex angle also passes through the rotation axis 704. Therefore, the design must satisfy the equation β/2+12.4138+β/2=45. Solving yields an apex angle of β=32.5862 degrees.

The next prism 917 moving counterclockwise on the disk 760 from prism 806 is tilted slightly by an angle +α so its bisecting line 17L does not pass through the center of rotation 704 of the disk. As a result, the included angle for the sector formed by prisms 806 and 917 is (β/2+α)+12.4138+β/2=45+α. The next prism 916 is once again aligned with the rotation center 704, so the included angle for the sector formed by prisms 916 and 917 is (β/2−α)+12.4138+β/2=45−α. The next prism is tilted by +2α, followed by an aligned prism, and then a prism tilted by +3α, followed by another aligned prism, etc. This geometry is maintained around the periphery of disk 760. This specific arrangement produces twenty nine deflection angles that vary over the range of −15 degrees to +15 degrees relative to the nominal 90 degree magnitude. Note that this approach uses an odd number of sectors where every other (approximately) prism is aligned and the alternate prisms are tilted by angles α, 2α, 3α, etc.

Other numbers of sectors and different deflection angle patterns can be produced by variants of this specific geometry. In addition, other rotation schemes are possible that produce the same angular deflection but do not produce them in monotonically increasing order. As another example, the rotating component could have an even number of sectors and prisms, with every other prism aligned and the alternate prisms tilted by angles α/2, 3α/2, 5α/2, etc. This would produce a set of angular deflections centered around a nominal magnitude, but without producing a deflection actually at the nominal magnitude.

In another approach, the rotation scheme causes the angular deflections to be arranged in a sequence such that the final delivered spots are not produced in sequential order. In other words, if the pattern is an array of spots 1, 2, 3, . . . 29, the sectors may be designed to generate the spots in an order other than sequentially from 1 to 29. For certain applications, producing adjacent spots within a short period of time can cause thermal coupling between the irradiated regions, and this can be deleterious to proper treatment. By arranging the prisms appropriately, the spots can be delivered such that temporally successive spots are spatially separated from each other while still delivering the full pattern of spots.

There is another geometric symmetry that is beneficial for some applications. Certain applications benefit by image patterns that are arranged to be in a zig-zag rather than a straight line geometry. For example, in some biologic applications, if the image spots are arranged along a straight line and high irradiance levels are present, the irradiation may accidentally cut tissue in the manner of a laser scalpel. Depositing the image spots in a zig-zag pattern substantially reduces the propensity for cutting or for undesirable thermal damage to biologic tissue while still permitting the thermal treatment level to be delivered. To achieve the zig-zag pattern, the prisms in the above-described geometry that have a rotation angle α applied to them can also have an orthogonal tilt angle applied to produce the lateral spot displacements used to produce a zig-zag geometry.

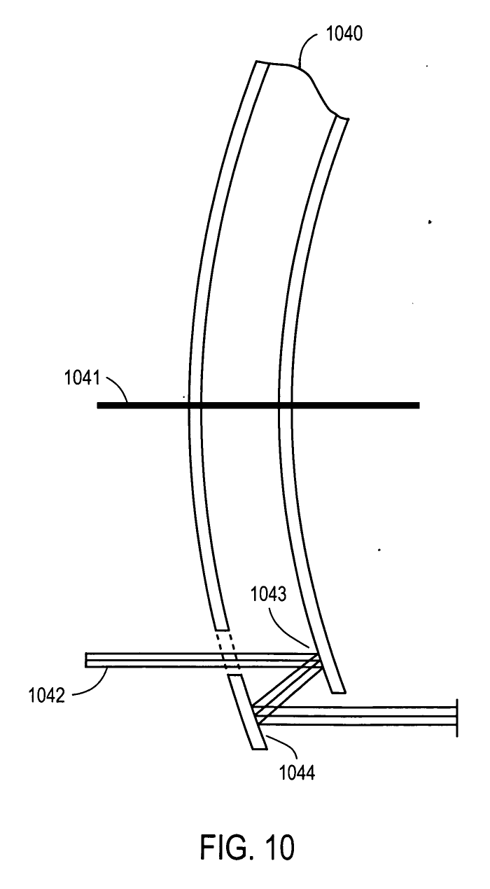

FIG. 10 is a view of another optical pattern generator according to the invention, where the direction of propagation for the incident optical beam 1042 has a substantial component in a direction normal to the plane of rotation. This pattern generator also uses a single rotating component 1040 with rotation axis 1041 to generate the pattern of interest. In this example, the disk 1040 supports reflective segments 1043, 1044 that are rotated through the incident optical beam. The segments have parent optical surfaces that are rotationally symmetric, with their optical axes coincident with the rotation axis 1041 of the rotating component. FIG. 10 shows the large, parent optical surfaces with their smaller reflective segments 1043, 1044 where the beam 1042 reflects twice and then exits the pattern generator. In FIG. 10, the rotating component includes a disk 1040 with pairs of opposing reflective surfaces 1043, 1044 for each sector, where different sectors may contain reflective surfaces with different radii of curvature such that the exiting beams will be displaced at different angles for each sector, but retain the PSD condition. Because surfaces 1043, 1044 are rotationally symmetric and are rotated about their optical centerlines, both of the surfaces 1043, 1044 that intersect with the optical beam are spatially invariant with respect to rotation. The radii of the two reflective segments 1043, 1044 and their axial separation are chosen to keep the system approximately afocal for all segments while simultaneously varying the output beam angle.

Examples of other types of optical pattern generators include galvanometers, acousto-optic elements, electro-optic elements, piezoelectric elements, micro-electromechanical devices (MEMs), and rotating elements (e.g., rotating mirrors and prisms). More detail regarding the single rotating component optical pattern generators described above with regard to FIGS. 7-10 and other examples of optical pattern generator embodiments are included in U.S. patent application Ser. No. 11/158,907, “Optical Pattern Generator Using a Single Rotating Component,” filed Jun. 20, 2005, which is incorporated by reference herein in its entirety.

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents.

What is claimed is:

1. An apparatus for achieving beneficial effects in a target tissue within a human body, the apparatus comprising:
an optical pattern generator for directing an optical beam to generate a predetermined irradiation pattern at the target tissue, the irradiation pattern creating a plurality of microscopic treatment zones separated by untreated target tissue; and
a probe for maintaining an optical channel within the human body for delivering the optical beam to the target tissue.

2. The apparatus of claim 1 wherein the microscopic treatment zones have a width of between approximately 20 and 200 μm.

3. The apparatus of claim 1 wherein a volume of untreated target tissue is greater than a volume of microscopic treatment zones.

4. The apparatus of claim 1 wherein the irradiation pattern comprises an annular pattern.

5. The apparatus of claim 1 wherein the irradiation pattern comprises a conical pattern.

6. The apparatus of claim 1 wherein the irradiation pattern comprises a plurality of deblurred spots.

7. The apparatus of claim 1 wherein the irradiation pattern comprises an irregular pattern of illuminated spots.

8. The apparatus of claim 1 wherein the probe comprises an optical window in direct contact with the target tissue, the optical beam passing through the optical window.

9. The apparatus of claim 8 wherein the optical window is thermally conductive.

10. The apparatus of claim 1 wherein the probe is an endoscopic probe.

11. The apparatus of claim 1 wherein the probe is an arthroscopic probe.

12. The apparatus of claim 1 wherein the probe is a catheter probe.

13. The apparatus of claim 1 further comprising:
a controller coupled to monitor motion of the probe and for controlling the optical beam and/or the optical pattern generator based on the motion of the probe.

14. The apparatus of claim 1 further comprising:
a controller for controlling at least one of the following parameters for the optical beam:
treatment zone pattern, exposure period, and energy density distribution.

15. The apparatus of claim 1 further comprising:
a sensor for monitoring treatment of the target tissue; and
a controller coupled to the sensor for controlling irradiation of the target tissue based on the monitored treatment.

16. The apparatus of claim 1 wherein the optical pattern generator comprises:
a single rotatable component having a plane of rotation and a rotation axis, the rotatable component comprising a plurality of deflection sectors arranged in a pattern around the rotation axis, wherein each sector deflects the optical beam as the sector rotates through the optical beam to generate the predetermined irradiation pattern at the target tissue.

17. The apparatus of claim 16 wherein the deflection sectors are arranged approximately in a circle centered on the rotation axis, and the sectors are substantially self-compensating with respect to a rotation of the rotatable component and are substantially spatially invariant with respect to a wobble of the rotatable component.

18. The apparatus of claim 16 wherein each sector is adapted to deflect the incident optical beam by a substantially constant angular deflection that is primarily in the plane of rotation.

19. The apparatus of claim 16 wherein, for a majority of the deflection sectors on the rotatable component, the sector comprises a pair of opposing reflective surfaces that have a substantial component in the plane of rotation for deflecting the incident collimated optical beam toward different points in the irradiation pattern.

20. The apparatus of claim 16 wherein the rotatable component comprises a plurality of discrete structures arranged approximately around the rotation axis adjacent to the sectors, each discrete structure having at least two reflective faces, and reflective faces from adjacent structures form opposing reflective surfaces for the sectors.

21. The apparatus of claim 20 wherein the discrete structures are prisms.

22. The apparatus of claim 20 wherein the discrete structures are prisms and approximately every other prism is aligned so that a bisection line of an apex angle of the prism also runs through the rotation axis.

23. The apparatus of claim 1 wherein the optical pattern generator comprises:
two counter-rotating disks for deflecting an incident optical beam to generate the predetermined irradiation pattern at the target tissue.

24. The apparatus of claim 23 wherein the irradiation pattern comprises an annular pattern and the apparatus further comprises:
a pyramidal polygon having N facets, wherein each counter-rotating disk has N facets and each pair of facets on the counter-rotating disks deflects the incident optical beam to a corresponding facet on the pyramidal polygon.

25. The apparatus of claim 23 wherein the irradiation pattern comprises a plurality of spots, the counter-rotating disks have pairs of corresponding facets and each pair of corresponding facets generates one of the spots and the spot is substantially stationary as the pair of facets rotates through the incident optical beam.

26. The apparatus of claim 23 wherein the two counter-rotating disks comprise pairs of corresponding facets and one facet of a pair of corresponding facets behaves as a positive lens and the other facet behaves as a negative lens.

27. The apparatus of claim 26 wherein the centers of rotation of the two counter-rotating disks is separated by a distance L and the optical centers of the positive lens and the negative lens are also separated by the distance L.

28. The apparatus of claim 26 wherein the centers of rotation of the two counter-rotating disks is separated by a distance L and the optical centers of the positive lens and the negative lens are separated by a distance approximately equal to L but not exactly equal to L, in order to correct for residual cross-scan angular displacement of the deflected optical beam.

29. The apparatus of claim 26 wherein at least one of the facets includes an aspheric surface for correcting for residual cross-scan angular displacement of the deflected optical beam.

30. The apparatus of claim 26 wherein the positive lens and the negative lens have slightly different focal lengths in order to correct for residual cross-scan angular displacement of the deflected optical beam.

31. A method for achieving beneficial effects in a target tissue within a human body, the method comprising:
generating an optical beam;
directing the optical beam to generate a predetermined irradiation pattern at the target tissue, the irradiation pattern creating a plurality of microscopic treatment zones separated by untreated target tissue;
maintaining an optical channel within the human body; and
delivering the optical beam to the target tissue via the optical channel.
